(19) United States
(12) Patent Application Publication
(10) Pub. No.: US 2011/0290248 A1
(43) Pub. Date: Dec. 1, 2011

(54) Aerosol Generator

(76) Inventor: Steven Michael Schennum, Plainfield, IL (US)

(21) Appl. No.: 12/787,258

(22) Filed: May 25, 2010

(51) Int. Cl. A61M 15/06

(52) U.S. Cl. 128/202.21

(57) ABSTRACT

An aerosol generator has a generally cylindrical body with a mouthpiece at one end a removable canister received within the body, the canister containing pressurised fluid, an interior, continuous canister valve and a depressible discharge tube that releases the fluid into the body. An electrically operable valve controls the discharge of the fluid from the canister towards the mouthpiece, and a nozzle forms an aerosol of the fluid that discharges from the canister. A pressure switch switches in response to a pressure reduction at the mouthpiece and supplies electrical power from internal batteries to the valve to produce a discharge of the fluid through the valve to create an aerosol in the mouthpiece for the consumer.

### FIELD OF THE INVENTION

This invention relates to a portable, aerosol generator which may be hand held and used to deliver aerosol to the mouth of a consumer, for example, but not exclusively an aerosol containing nicotine.

### BACKGROUND

A nicotine dispensing aerosol device is disclosed in U.S. Pat. No. 4,945,929, which simulates a smoking article such as a cigarette, without having to burn tobacco.

### SUMMARY OF THE INVENTION

The invention provides an aerosol generator that is electrically actuated by means of a pressure switch and a valve. In an embodiment of the invention the aerosol device has a body with a mouthpiece, the body being configured to receive a supply of pressurised fluid to be discharged towards the mouthpiece as an aerosol, and an electrically operable valve is provided to control the discharge of the fluid towards the mouthpiece. The pressure switch is configured to switch electrical power to the valve to produce a discharge of the fluid through the valve in response to a pressure reduction at the mouthpiece.

### BRIEF DESCRIPTION OF THE DRAWINGS

In order that the invention may be more fully understood, an embodiment thereof will now be described by way of illustrative example with reference to the accompanying drawings in which:

FIG. 1 is a side view of an aerosol generator;

FIG. 2 is a mouth end view of the generator shown in FIG. 1;

FIG. 3 illustrates the aerosol generator when hand held for use by a consumer;

FIG. 4 is a schematic illustration of the major components of the generator when partially disassembled;

FIG. 5 is a longitudinal sectional view of the generator when partially disassembled;

FIG. 6 is a longitudinal sectional view of the generator corresponding to FIG. 5, when fully assembled;

FIG. 7 is an exploded perspective view of the generator with the components at its proximal, mouth end shown in more detail;

FIG. 8 is an exploded perspective view of the generator with the components intermediate the proximal and distal ends shown in more detail;

FIG. 9 is an exploded perspective view of the generator with the components at its distal end shown in more detail;

FIG. 10 is a schematic circuit diagram of the electrical components of the aerosol generator;

FIG. 11a is an enlarged partial sectional view of the proximal, mouth end, showing the solenoid actuated valve for creating the aerosol, when closed;

FIG. 11b corresponds to the view of FIG. 11a but showing the solenoid actuated valve driven open to create the aerosol;

FIG. 12 is an enlarged, broken away view of the distal end illustrating the puff actuated pressure switch in more detail;

FIG. 13 an enlarged, broken away view of components in a region between the proximal and distal ends, illustrating a pressure communication passageway to the pressure switch;

FIGS. 14a and 14b illustrate the pressure switch in its inoperative and operative states respectively;

FIG. 15 is a further broken away view of the mouth end showing the pressure communication passageway extending around the solenoid operated valve;

FIG. 16 illustrates the interior arrangement of the solenoid operated valve within the mouth end shells and showing the pressure communication passageway extending around the solenoid operated valve; and

FIG. 17 is a perspective view of the battery compartment.

### DETAILED DESCRIPTION

### Overview

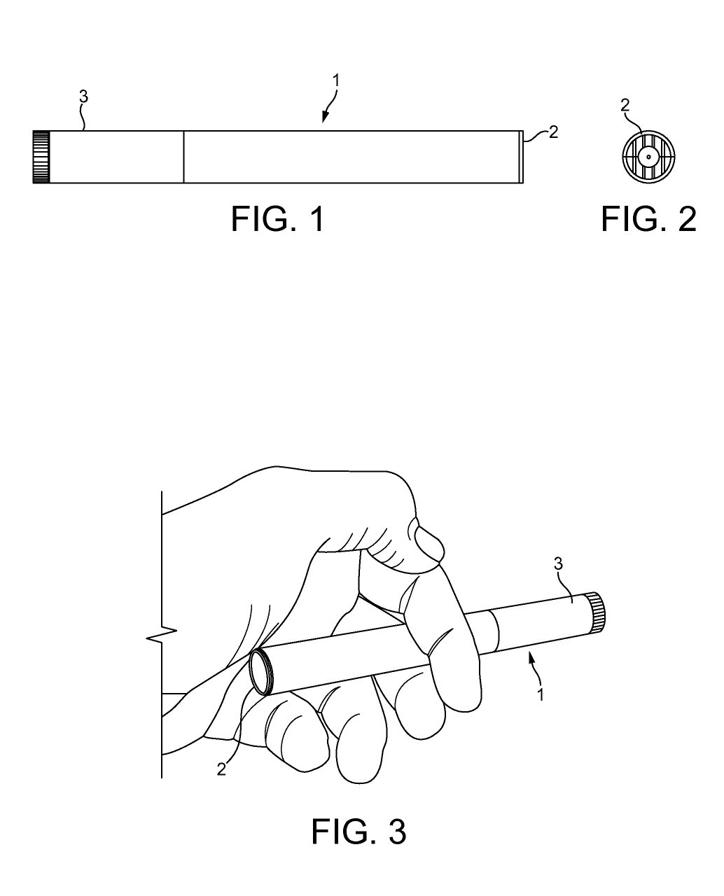

An aerosol generator 1 is illustrated in FIGS. 1 and 2, which is generally cylindrical and may be held between the fingers of a consumer's hand as illustrated in FIG. 3, to simulate a smoking article such as a cigarette. The generator 1 has a proximal, mouth end 2 and a distal end 3, and delivers an aerosol to the mouth end 2. The aerosol may contain nicotine.

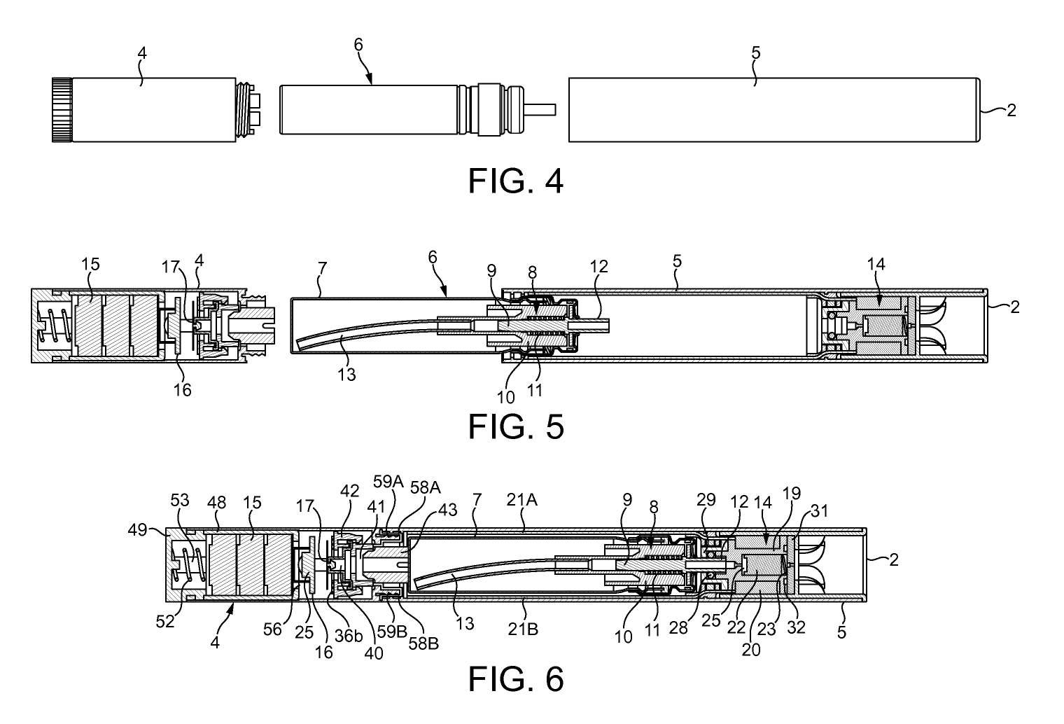

Referring to FIG. 4, the cylindrical body of the generator comprises a distal end housing 4 that contains batteries, control circuit and a pressure actuated switch to be described in more detail later. The distal end housing 4 is releasably attached coaxially to a cylindrical main housing 5. A canister 6 containing pressurised fluid for forming an aerosol is fitted into the main housing 5 and retained in place by the distal end housing 4.

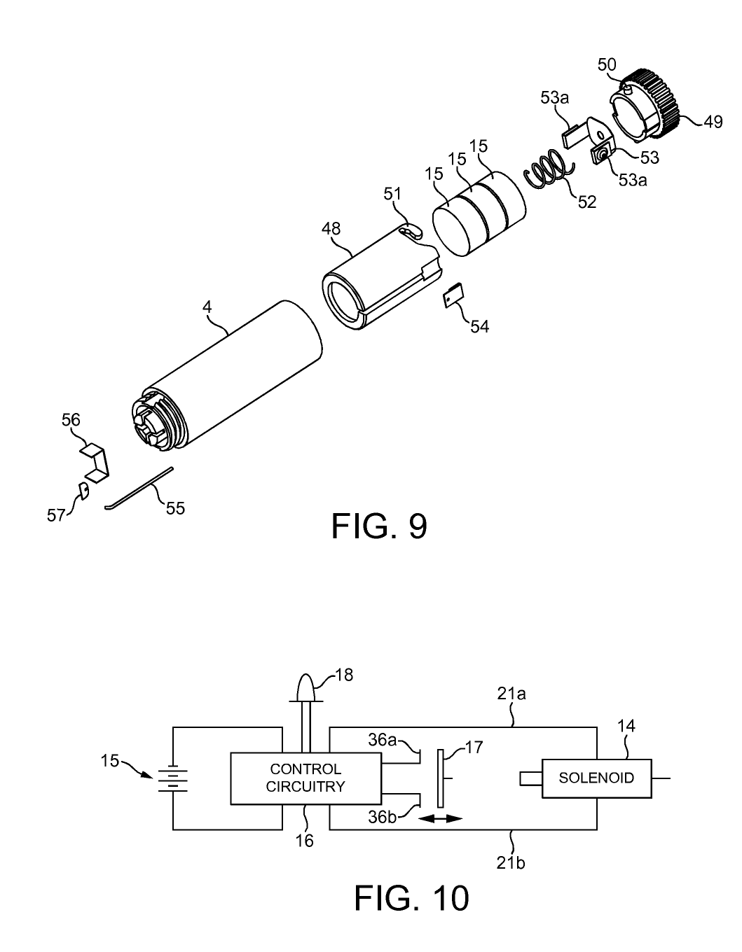

The structure of the aerosol generator 1 will now be described in detail with reference to FIGS. 5 to 9. As shown in FIGS. 5 and 6, the canister 6 comprises a generally cylindrical body 7 and an integral valve 8 which includes a valve stem 9 slidably mounted in a valve seat 10, biased to be normally closed by spring 11. The valve stem 9 has an axial discharge tube 12 through which pressurised liquid from within the container is discharged when depressed axially inwardly of the canister, the liquid being fed through inlet tube 13 from within the canister 6 to the valve 8. The valve 8 is a so-called continuous valve, which, when the valve stem 9 is pushed inwardly to open the valve, discharges fluid from the canister continuously until empty or until the valve stem is released to close the valve.

Canister 6 can be interchanged by removal of the distal end housing 4 as can be seen from FIGS. 4, 5 and 6. The canister 6 may be charged with a nicotine containing liquid and propellant such as HFA although other flavourants and propellants may be used.

When the canister 6 is installed as shown in FIG. 6, it is driven against a solenoid operated valve 14. When installed, the discharge tube 12 is pressed axially inwardly of the canister 6 to open the valve 8 against the force of its interior spring 11 so that the discharge of liquid from the canister is controlled by the solenoid operated valve 14.

As shown in FIGS. 5 and 6, the distal end housing 4 contains batteries 15 which provide electrical power to circuitry on a printed circuit board (PCB) 16, which controls the supply of the electrical power to the solenoid operated valve 14 in response to actuation of a pressure switch 17 that is responsive to changes in pressure in the mouthpiece 2.

FIG. 10 illustrates the electrical circuitry of the aerosol generator schematically. The batteries 15 are coupled to the control circuitry on printed circuit board 16 and electrical power is fed from the control circuitry to the solenoid operated valve 14 under the control of the pressure actuated switch 17. The control circuitry 16 may comprise an integrated circuit digital processor or may comprise an analog circuit. The circuitry 16 is responsive to operation of the pressure switch 17 to supply power from the battery 15 to the solenoid operated valve 14 for a predetermined period so as to generate a puff of aerosol from liquid discharged from the canister 6. The switch 17 is actuated by the user drawing on the mouthpiece 2 and the pressure reduction is communicated along the cylindrical housing 5 to the switch 17 through passageways to be described in more detail hereinafter.

The control circuitry 16 may be configured so that once a first puff has been taken by the consumer, the solenoid operated valve 14 is inhibited from producing a further puff for a predetermined period of time. The control circuitry 16 may perform other functions and for example may include a battery sensor that indicates when the batteries are becoming discharged, to operate a light emitting diode 18 or similar indicator illustrated in FIG. 10 (not illustrated elsewhere in the drawings). Also, it will be understood that whilst the device shown in the drawings utilises three button cell batteries 15, other battery configurations may be utilised, including rechargeable batteries.

### Solenoid Operated Valve

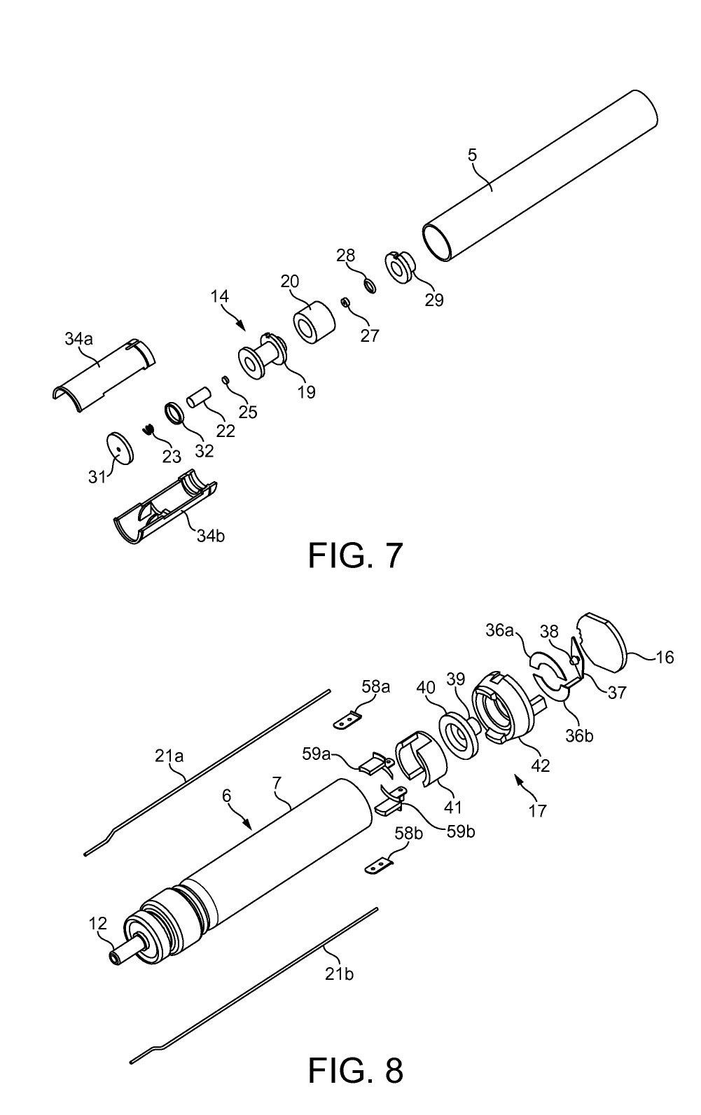

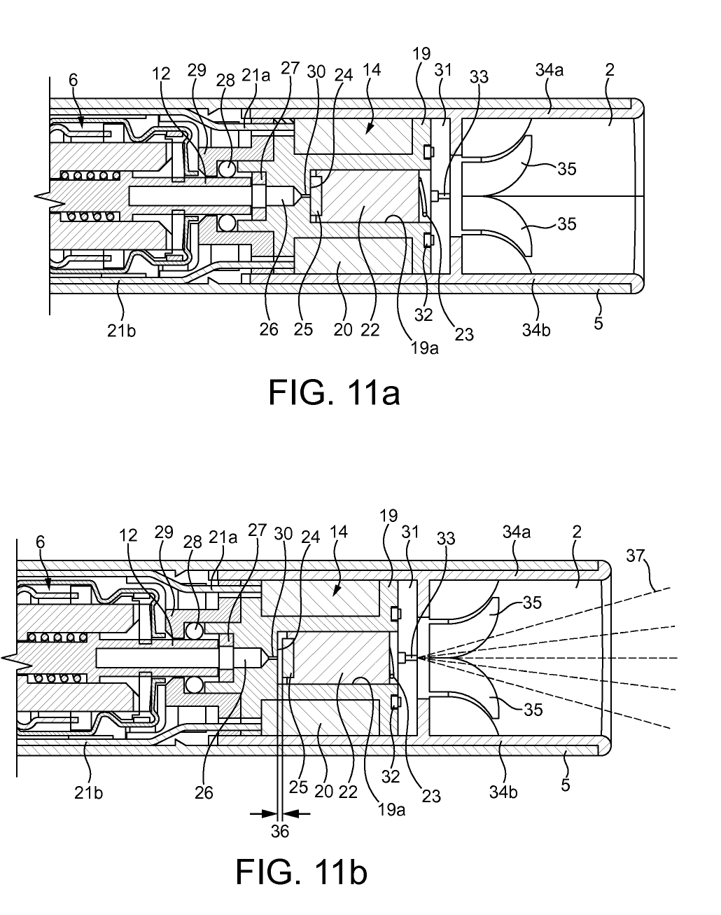

The construction and operation of the solenoid operated valve 14 will now be described in more detail with reference to FIGS. 7 and 11. The valve 14 includes a solenoid body 19 in the form of a bobbin that receives a solenoid coil 20. Wires 21a, 21b run along the interior of the cylindrical housing 5 for supplying electrical power to the solenoid coil 20.

The bobbin 19 includes a longitudinal passageway comprising a central bore 19a that receives a valve member comprising a solenoid plunger 22 biased by spring 23 against a valve seat 24. The plunger 22 is slidably received in the bore 19a with a small clearance to allow the passage of fluid through the bore 19a from the canister around the plunger when the valve is open, as will be explained in more detail later. A valve seal 25 is fixed to the plunger 22 to seal against the valve seat 24 when the valve 14 is closed. The bobbin 19 includes at its distal end a stepped bore 26 which receives the discharge tube 12 of the canister 6. A gasket seal 27 is disposed between the end of the discharge tube 12 and the stepped bore 26 to provide a seal. Additionally, an O-ring 28 is configured around the discharge tube 12 and is retained by a circular valve inlet member 29. The stepped bore 26 extends into an inlet bore 30, which is normally closed by the solenoid plunger 22 and seal 25.

The valve 14 also includes a nozzle outlet 31 in the form of a circular plate which is sealed against the proximal end of the bobbin 19 by a circular outlet gasket 32. The nozzle outlet 31 includes an axial outlet orifice 33 to discharge aerosol into the mouthpiece 2.

The solenoid valve and nozzle assembly is received within generally cylindrical housing shells 34a, 34b which when assembled together can be slide as a tubular unit into the housing 5 from the proximal, mouthpiece end 2. The housing shells 34a, b include fins 35 for structural support.

When the canister 6 is installed in the housing 5, it is pressed against the bobbin 19 and gasket seal 27 so that the discharge tube 12 of the canister is pressed inwardly against force of spring 11 with the result that fluid under pressure is applied from the canister 6 against the valve seat 24. When the valve 14 is unenergised as illustrated in FIG. 11a, the solenoid plunger 22 is driven by spring 23 against the valve seat 24 to close the valve and the seals 25, 27 and 28 ensure that no fluid leakage occurs.

When the solenoid valve 14 is actuated, the magnetic field produced by the coil 20 urges the solenoid plunger 22 towards the proximal end 2 as illustrated in FIG. 11b, against the force of the spring 23, so as to open the valve and produce a spacing 36 between the distal end of the solenoid plunger 22 and seal 25, and the valve seat 24. Fluid from the canister 6 can then pass through the discharge tube 12 and the inlet orifice 30, so as to pass through the gap between the cylindrical outer surface of the solenoid plunger 22 and the central bore 19a of bobbin 19, to the orifice 33 in the nozzle outlet 31. As a result, an aerosol spray illustrated by dotted lines 37 is directed to the mouthpiece 2.

### Pressure Sensitive Switch

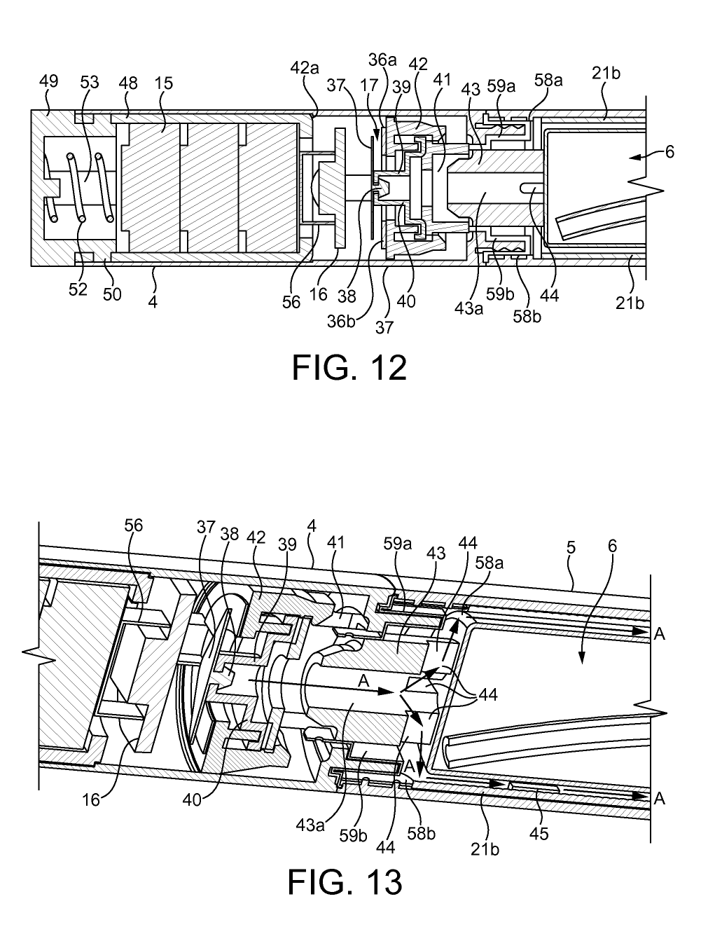

Referring to FIGS. 8, 12, 13 and 14, the pressure sensitive switch 17 is located between the canister 6 and the batteries 15. The switch 17 comprises a pair of semicircular switch contacts 36a, 36b (also shown in FIG. 10) which are spaced apart and each electrically connected to the control circuit on PCB 16. The switching contacts 36a, 36b are bridged selectively by a movable, electrically conductive bridge contact 37 which has a location lug 38 received in an axial extension 39 of a resilient diaphragm 40 sandwiched between a proximal and distal annular switch supports 41, 42. As shown in FIG. 13, the pressure switch assembly abuts an annular spacer member 43 that comprises the proximal end of the distal housing 4, which provides an end stop against the canister 6 when assembled.

The spacer member 43 includes an axial passageway 43a provided with radially extending slots 44. The main housing 5 includes ribs 45 that provide locating points for the canister 6 along its length so that an air passageway is provided around the canister extending from the radial slots 44 towards the mouthpiece 2.

When the consumer puffs on the mouthpiece 2, a pressure reduction occurs so that air travels in the direction of arrows A, producing a pressure differential across the diaphragm 40. As a result, the lug 39 on the diaphragm is moved axially towards the mouthpiece 2, pulling the bridge contact 37 into electrical connection with the switch contacts 36a, 36b. The contacts 36a, 36b are electrically connected to the control circuitry on PCB 16, which detects the electrical bridging of the contacts and in response, feeds a drive current to the solenoid operated valve 14 through conductors 21a, 21b and additional wires (not shown). As previously explained, the control circuit may operate the solenoid 14 for a predetermined time to achieve a particular duration of puff. Alternatively, the circuitry 16 may operate the solenoid for the duration that the consumer lowers pressure in the mouthpiece 2, so as to provide a continuous production whilst the consumer draws on the mouthpiece 2.

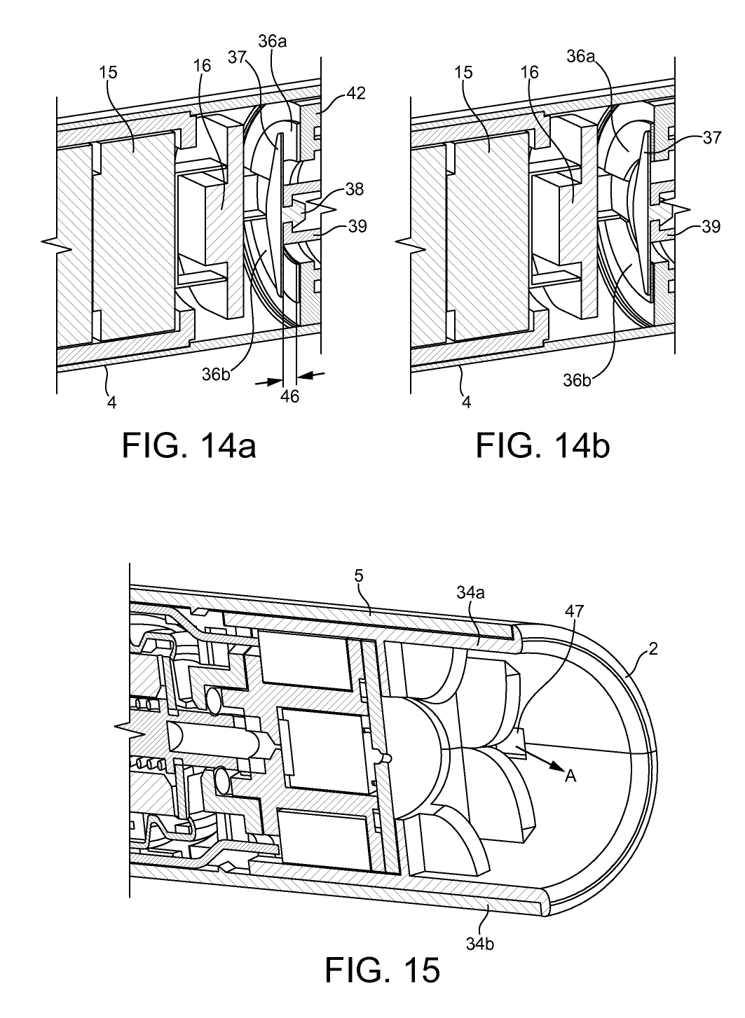

Thus, the negative pressure produced by the consumer results in the bridging contact 37 moving from the position shown in FIG. 14a spaced by a distance 46 from the contacts 36a, 36b, to the position shown in FIG. 14b in which the bridging contact 37 touches the switch contacts 36a, 36b.

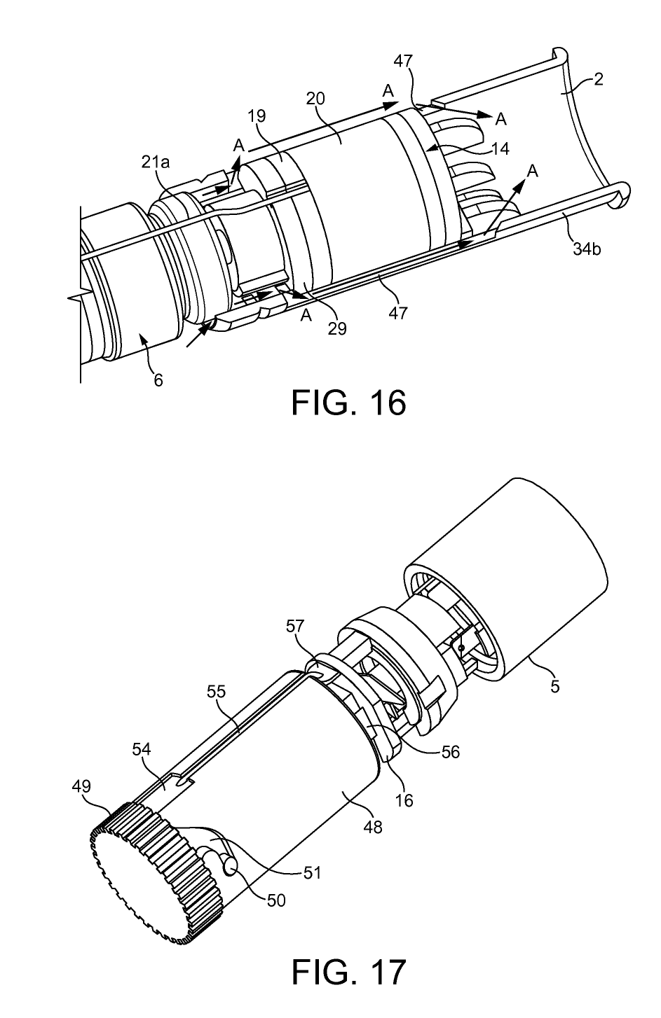

Referring to FIGS. 15 and 16, a passageway is provided in the annular space between the canister 6 and main housing 5 to allow negative pressure in the mouthpiece 2 to communicate with the pressure sensitive switch 17. To this end, the shells 34a, 34b are provided with cutaway portions 47 to provide axially extending passageways that allow air to flow in the direction of arrows A when the consumer sucks on the mouthpiece 2. The volume of the passageway between the mouthpiece and the switch 17 is made sufficiently small that the negative pressure produced by sucking the mouthpiece is not degraded significantly.

### Battery Compartment

Referring to FIGS. 5, 6, 9 and 17, the batteries 15 are received in a stack within a generally cylindrical battery holder 48 having a removable closure member 49. In this example, the closure member 49 is located by lugs 50 that are received in corresponding slots 51 to provide a bayonet mounting. The batteries 15 are located by a spring 52 which provides an electrical connection to a distal battery contact 53 that has axially extending tongues 53a that engage with battery contact 54 fixed to the exterior of the battery holder 48. The contact 54 is electrically connected to battery wire 55 that is mounted axially along the battery holder 48 and provides an electrical connection to the circuitry on PCB 16. A proximal battery contact 56 is provided which, as shown clearly in FIG. 13, is electrically connected to the proximal end of the stack of batteries 15. The proximal battery contact 56 is mounted directly on the PCB 16. A contact 57 also directly mounted on the PCB 16 provides an electrical connection between a proximal end of battery wire 55 and PCB 16.

As illustrated in FIG. 12, the assembled battery compartment is push-fitted into the distal end housing 4 along with the pressure switch 17 so as to abut an interior, annular step 42a in the housing 4.

FIG. 17 illustrates the distal end of the aerosol generator with the distal end of the housing 4 removed. The electrical conductors 21a, 21b that extend from the solenoid operated valve 17 terminate in distal end contacts 58a, 58b mounted within the distal end of the main housing 5. The contacts 58a, 58b electrically connect to contacts 59a, 59b mounted in the proximal end of the distal housing 4, within its end piece 42 and are so configured that when the distal end housing 4 is screwed into place on the main housing 5, the contacts 58a, 58b mate with the contacts 59a, 59b respectively. The contacts 59 are each electrically connected to the PCB 16 by connecting wires (not shown) although it will be appreciated that metallisation contacts may be formed on the interior of the housing 4, if desired, instead of separate wiring.

### Operation

In use, a consumer draws on the mouthpiece 2 in order to reduce the ambient pressure in the mouthpiece, which draws air from the vicinity of the pressure switch 17 in the direction of arrows A, causing the pressure switch to move from the position shown in FIG. 14a to that shown in FIG. 14b. As a result, the control circuitry on PCB 16 shown in FIG. 10 energises the solenoid operated valve 14 to release a puff of aerosol to the consumer through the mouthpiece.

Many modifications and variations will be evident to those skilled in the art. For example, the canister 6 may be provided with an alignment mechanism to arrange the inlet tube 13 to be configured downwardly in normal use to ensure that the entire contents of the canister 6 can be consumed. For example, the supply tube of the pressurised canister may be of flexible construction and provided with a weighted end as to always assume the lowest position in the canister orientation and allow for complete consumption of the canister contents.

Furthermore, the circuitry on PCB 16 may include an LED to indicate when the pressure switch 17 is actuated. Many other modifications and variations falling within the scope of the claims will be evident to those skilled in the art.

Also whilst the described examples of generator device are generally cylindrical with a circular cross section, other cross sectional shapes can be used, such as rectangular or triangular.

Furthermore, although the canister is described as an interchangeable element to allow the supply of liquid to be replenished, an integral pressurised supply vessel may be provided in the device, so that the device can be used multiple times and then discarded once the liquid supply has been exhausted from the vessel. In a modification, an inlet valve may be provided to allow the supply to be replenished from an exterior, pressurised source.

The mouth end can be configured differently from that shown in the Figures. For example the mouth end could be a tapered cylindrical shape with flat piece for easy positioning and orientation cue in the mouth. Also, textured or rough finishes can be applied to the exterior surface of the mouthpiece to stimulate sensation in the lips.

Also, it will be appreciated that the device may include alternative electrical power sources to the batteries described with reference to the drawings, for example rechargeable batteries or a portable fuel cell.

Inventive variations and aspects of the disclosure may include other non-limiting embodiments. For example, in one embodiment, an aerosol generator may comprise a body having a mouthpiece, the body being configured to receive a supply of pressurised fluid to be discharged towards the mouthpiece as an aerosol, an electrically operable valve to control the discharge of the fluid towards the mouthpiece, and a pressure switch configured to switch electrical power to the valve to produce a discharge of the fluid through the valve in response to a pressure reduction at the mouthpiece.

In one implementation, the generator may include a nozzle to form the aerosol from the fluid discharged from the valve.

In one implementation, the body may be elongate and may be configured to be held between the fingers of a hand. In one implementation, the body may include a compartment to receive a supply of the pressurised fluid. In one implementation, the compartment may comprise a canister compartment that is configured to receive a canister containing the pressurised fluid with an interior canister valve and a depressible discharge tube that releases the fluid into the body, canister compartment being configured to hold the interior canister valve open when fitted into the body. In one implementation, the generator may include the canister. In one implementation, the body may include a battery compartment to receive a battery to power the valve.

In one implementation, the generator may include a generally cylindrical main body housing and a distal end housing releasably attached to and coaxial with the main body housing.

In one embodiment, the generator may include a battery compartment and the pressure switch in the distal end housing. In one implementation, the pressure switch may include a diaphragm moveable in response to said pressure reduction at the mouthpiece, and an electrically conductive switch contact moved by the diaphragm in response to the pressure reduction.

In one implementation, the generator may include an electrical circuitry responsive to operation of the pressure switch to supply electrical power to the valve. In one implementation, the circuitry is operable to supply the electrical power to the valve for a predetermined time following actuation of the pressure switch. In another implementation the circuitry may be operable to inhibit supply of the electrical power to the valve for a predetermined time following operation of the switch.

In one implementation, the electrically operable valve may comprise a solenoid operated valve. In one implementation, the valve may include a bobbin with a longitudinal passageway therein, a valve seat in the passageway, an inlet to supply the fluid to the passageway, a valve member moveable in the passageway to engage the valve seat, and an electrical coil to be energised for moving the valve member to open and close the valve.

In another embodiment, an aerosol generator may comprise a generally cylindrical body having a mouthpiece and a canister compartment, a canister removably received within the canister compartment, the canister containing pressurised fluid, an interior canister valve and a depressible discharge tube that releases the fluid into the body, the canister compartment being configured to hold the interior canister valve open, an electrically operable valve to control the discharge of the fluid from the canister towards the mouthpiece, a nozzle to form an aerosol of the fluid that discharges from the canister, and a pressure switch configured to switch electrical power to the valve to produce a discharge of the fluid through the valve in response to a pressure reduction at the mouthpiece.

In one implementation, the generator may include a battery compartment at an end of the housing distal from the mouthpiece, the pressure switch being located between the canister and the battery compartment.

1. An aerosol generator comprising:
a body having a mouthpiece, the body configured to receive a supply of pressurised fluid discharged towards the mouthpiece as an aerosol;
an electrically operable valve configured to control the discharge of the fluid towards the mouthpiece; and
a pressure switch configured to switch electrical power to the valve and produce a discharge of the fluid through the valve in response to a pressure reduction at the mouthpiece.

2. The generator according to claim 1, further comprising a nozzle configured to form the aerosol from the fluid discharged from the valve.

3. The generator according to claim 1, wherein the body is elongate and configured to be held between the fingers of a hand.

4. The generator according to claim 1, wherein the body includes a compartment configured to receive a supply of the pressurised fluid.

5. The generator according to claim 4, wherein the compartment comprises a canister compartment configured to receive a canister containing the pressurised fluid with an interior canister valve and a depressible discharge tube that releases the fluid into the body, the canister compartment configured to hold the interior canister valve open when fitted into the body.

6. The generator according to claim 5, further comprising the canister.

7. The generator according to claim 1, wherein the body includes a battery compartment configured to receive a battery to power the valve.

8. The generator according to claim 1, further comprising a generally cylindrical main body housing and a distal end housing releasably attached to and coaxial with the main body housing.

9. The generator according to claim 8, including a battery compartment and the pressure switch in the distal end housing.

10. The generator according to claim 1, wherein the pressure switch includes a diaphragm moveable in response to said pressure reduction at the mouthpiece, and an electrically conductive switch contact moved by the diaphragm in response to the pressure reduction.

11. The generator according to claim 1, including electrical circuitry responsive to operation of the pressure switch configured to supply electrical power to the valve.

12. The generator according to claim 11, wherein the circuitry is operable to supply the electrical power to the valve for a predetermined time following actuation of the pressure switch.

13. The generator according to claim 11, wherein the circuitry is operable to inhibit supply of the electrical power to the valve for a predetermined time following operation of the switch.

14. The generator according to claim 1, wherein the electrically operable valve comprises a solenoid operated valve.

15. The generator according to claim 14, wherein the valve comprises a bobbin with a longitudinal passageway, a valve seat in the passageway, an inlet to supply the fluid to the passageway, a valve member moveable in the passageway to engage the valve seat, and an electrical coil to be energised for moving the valve member to open and close the valve.

16. An aerosol generator, comprising:
a generally cylindrical body having a mouthpiece and a canister compartment;
a removable canister received within the canister compartment, the canister containing pressurised fluid, an interior canister valve and a depressible discharge tube that releases the fluid into the body, the canister compartment being configured to hold the interior canister valve open;
an electrically operable valve configured to control discharge of the fluid from the canister towards the mouthpiece;
a nozzle configured to form an aerosol of the fluid that discharges from the canister; and
a pressure switch configured to switch electrical power to the valve and produce the discharge of the fluid through the valve in response to a pressure reduction at the mouthpiece.

17. The generator according to claim 16, including a battery compartment at an end of a housing distal from the mouthpiece, the pressure switch being located between the canister and the battery compartment.
